(19) United States
(12) Patent Application Publication
(10) Pub. No.: US 2026/0160755 A1
(43) Pub. Date: Jun. 11, 2026

(54) METHOD FOR ENHANCED DETERMINATION OF ANALYTE CONCENTRATION IN BODILY FLUID

(71) Applicant: Roche Diabetes Care, Inc., Indianapolis, IN (US)

(72) Inventors: Max Berg, Mannheim (DE); Fredrik Hailer, Mannheim (DE); Bernhard Limburg, Mannheim (DE)

(21) Appl. No.: 19/243,620

(22) Filed: Jun. 19, 2025

Related U.S. Application Data
(63) Continuation of application No. PCT/EP2023/086778, filed on Dec. 20, 2023.

(30) Foreign Application Priority Data
Dec. 22, 2022 (EP) 22 216 107.7

(51) Int. Cl. G01N 33/52; G01N 21/77; G01N 21/81

(52) U.S. Cl. CPC G01N 33/521; G01N 21/81; G01N 2021/7759

(57) ABSTRACT

An analytical method for determining a concentration of an analyte in a bodily fluid uses a mobile device having a camera. The camera captures an image of a test region having a sample of the bodily fluid applied thereto. The image includes at least a part of a hydrochromic indicator field that exhibits an optically detectable color change at a pre-determined threshold level of relative humidity rH_{threshold(m)}. From the color of the hydrochromic indicator field, an estimate value rHₑₛₜᵢₘₐₜₑ of the local relative humidity is derived and, based on the estimate value rHₑₛₜᵢₘₐₜₑ, an applicable range of relative humidity rH_{appl-range(n)} is selected. The concentration of the analyte from a color of the reagent test region in the image is determined, taking into account the applicable range of relative humidity rH_{appl-range(n)}.

### RELATED APPLICATIONS

This application is a continuation of International Application No. PCT/EP2023/086778, filed Dec. 20, 2023, which claims priority to European Application No. 22 216 107.7, filed Dec. 22, 2022, the entire disclosures of both of which are hereby incorporated herein by reference.

### BACKGROUND

This disclosure relates to a method of determining a concentration of an analyte in a bodily fluid, using at least one mobile device having a camera. Further, this disclosure relates to a mobile device having a camera for carrying out the method, to a kit comprising a mobile device having a camera, to computer programs and computer-readable storage media. The methods, mobile devices, computer programs and storage media specifically may be used in medical diagnostics, for example, in order to qualitatively or quantitatively detect one or more analytes in body fluids, such as for detecting glucose in blood or interstitial fluid.

In the field of medical diagnostics, in many cases, one or more analytes have to be detected in samples of a body fluid, such as blood, interstitial fluid, urine, saliva or other types of body fluids. Examples of analytes to be detected are glucose, triglycerides, lactate, cholesterol or other types of analytes typically present in these body fluids. According to the concentration and/or the presence of the analyte, an appropriate treatment may be chosen, if necessary.

Generally, devices and methods known to the skilled person make use of test elements comprising one or more test chemicals, which, in the presence of the analyte to be detected, are capable of performing one or more detectable detection reactions, such as optically detectable detection reactions. With regard to the test chemicals included in test elements, reference may be made, e.g., to J. Hoenes et al.: The Technology Behind Glucose Meters: Test Strips, Diabetes Technology & Therapeutics, Volume 10, Supplement 1, 2008, S-10 to S-26.

In analytical measurements, specifically analytical measurements based on color formation reactions, one technical challenge resides in the evaluation of the color change due to the detection reaction. Besides using dedicated analytical devices, such as handheld blood glucose meters, the use of generally available electronics such as smart phones and portable computers or other mobile devices has become more and more popular over the recent years.

As opposed to laboratory measurements and measurements performed by using dedicated analytical measurement devices, when using mobile computing devices such as smart phones, various additional influences need to be taken into account, such as, for example, lighting conditions and positioning aspects, which may be rather difficult to take into account. In order to improve the accuracy of results of analyte detection in these cases nonetheless, it is therefore beneficial to appropriately take into account any parameters known to be involved in the desired analyte detection or measurement.

For analytical measurements based on test chemicals, one such parameter usually is the temperature at which a reaction of an analyte with test chemicals takes place, see with regard to such test chemicals included in test elements, e.g., the above-cited reference to J. Hoenes et al. Furthermore, another such parameter which may affect test chemicals is humidity, specifically the ambient humidity at the location of the measurement, which often is referred to in terms of relative humidity.

When using mobile devices, one approach to take the temperature of a reaction on a test element into account is to provide a temperature sensor or a temperature indication area on the test strip itself. See, e.g., U.S. Pat. No. 9,778,200 B2 which, inter alia, describes a method for a portable computing device having an image sensor to read a reaction area on a test strip, which is located in a peripheral device, wherein the test strip may include a temperature indication area, and a specimen characteristic may be corrected based on the captured temperature indication area in the image. Also see EP 3 018 470 A1 which, inter alia, describes a method of a terminal measuring biometric information, comprising: receiving an image of a biosensor comprising a reagent pad on which a sample is collected, wherein the method may further comprise determining a temperature of the sample based on temperature information that is indicated by a temperature measurer that is attached to the reagent pad in the received image. Also see U.S. Publication No. 2013/0267032 A1 and EP 3 575 781 A2 which, inter alia, relate to a specimen test strip to detect a characteristic of an analyte in a specimen sample, wherein the specimen test strip may comprise a temperature indication area configured to correct a measurement of the characteristic of analyte.

In contrast to the aforementioned examples, wherein an impact of temperature may be targeted to be taken into account for an analytical measurement which involves the use of a test chemistry based on color formation and which involves the use of mobile computing devices such as smart phones, taking the impact of humidity into account in such a scenario has not been considered in the same way.

An example of a humidity-sensitive scenario in a non-diagnostic field is described in U.S. Publication No. 2021/0350689 A1 which, inter alia, relates to a workstation monitoring system, comprising a camera for receiving images, identifying a surface and a state for the surface (including, e.g., clean, dirty, contaminated, attended to, or not attended to), tracking behavior of an individual, movement of an object, and/or an occurrence for the surface that causes a change in the surface's state, and generating a trigger event signal based on the change in the surface's state; wherein a unique identifier may be embedded in a sticker, e.g., hidden under a hydrochromic ink when the sticker is dry and only visible when the ink is wet and therefore transparent, and said unique identifier may be read by a scanning mobile handheld device like a mobile phone.

EP 2 941 630 B1 relates to a specimen test strip to detect a characteristic of an analyte in a sample, comprising: a reaction area; and a color calibration area; wherein the test strip further comprises: a path for the sample, the path being defined by a capillary extending from a capillary entrance to the reaction area; a hole being defined through the capillary intermediate the capillary entrance and the reaction area; a top opening of the hole being covered by a first transparent film; and a bottom opening of the hole being covered by a second film. The test strip may further comprise a timer area to receive the sample and to change color linearly in response to the sample; and the test strip may further comprise a timer area to change color in response to light or humidity.

U.S. Publication No. 2022/0381773 A1 relates to an analytical method for determining a concentration of an analyte in a body fluid using a mobile device having a camera and a processor, wherein local temperature information at a current location of the mobile device is used for determining a correction temperature and/or a correction temperature function, which corrections are taken into account when determining the analyte concentration from an image, captured by the camera, based on a color formation reaction at a reagent test region of an optical test strip having the sample of the body fluid applied thereto. In the method described, humidity information from various sources may be considered.

Despite the advantages involved in using mobile computing devices for the purpose of performing an analytical measurement, one of the remaining technical challenges still is to appropriately take into account the impact of humidity with regard to a reaction involving an analyte to be detected and test chemicals comprised in test elements.

### SUMMARY

This disclosure teaches devices and methods which at least partially address the above-mentioned challenge. Specifically, devices and methods are disclosed which allow for an efficient mobile-based determination of a concentration of an analyte in a bodily fluid with reliable accuracy, however, with low effort for setup and implementation, which devices and methods particularly shall take into account an impact of humidity.

As used in the following, the terms “have,” “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B,” “A comprises B” and “A includes B” may both refer to a situation in which, besides B, no other element is present in A (i.e., a situation in which A solely and exclusively consists of B) and to a situation in which, besides B, one or more further elements are present in entity A, such as element C, elements C and D or even further elements.

Further, it shall be noted that the terms “at least one,” “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once. It shall also be understood for purposes of this disclosure and appended claims that, regardless of whether the phrases “one or more” or “at least one” precede an element or feature appearing in this disclosure or claims, such element or feature shall not receive a singular interpretation unless it is made explicit herein. By way of non-limiting example, the terms “camera,” “image,” and “indicator field,” to name just a few, should be interpreted wherever they appear in this disclosure and claims to mean “at least one” or “one or more” regardless of whether they are introduced with the expressions “at least one” or “one or more.” All other terms used herein should be similarly interpreted unless it is made explicit that a singular interpretation is intended.

Further, as used in the following, the terms “preferably,” “more preferably,” “particularly,” “more particularly,” “specifically,” “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of this disclosure, a computer-implemented, specifically in-vitro, analytical method for determining a concentration of an analyte in a bodily fluid is disclosed, the method comprising using a mobile device having at least one camera and, specifically, a processor. The method comprises the following steps which, as an example, may be performed in the given order. It shall be noted, however, that a different order is also possible. Further, it is also possible to perform one or more of the method steps once or repeatedly. Further, it is possible to perform two or more of the method steps simultaneously or in a timely overlapping fashion. The method may comprise further method steps which are not listed. The method comprises, in a first step i), receiving, specifically by the processor of the mobile device, at least one image captured by the camera of the mobile device. The image comprises at least a part of a reagent test region associated with an optical test element and/or associated with a color reference card, wherein the reagent test region has a sample of the bodily fluid applied thereto. Furthermore, the image comprises at least a part of at least one hydrochromic indicator field associated with the optical test element and/or associated with the color reference card. The hydrochromic indicator field exhibits at least one optically detectable color change at a pre-determined threshold level of relative humidity rH_{threshold(m)}.

The method further comprises:
ii) deriving, specifically by the processor of the mobile device, from the color of the hydrochromic indicator field in the image, an estimate value rHₑₛₜᵢₘₐₜₑ of the local relative humidity.

Step ii) further comprises selecting, specifically by the processor of the mobile device, based on the estimate value rHₑₛₜᵢₘₐₜₑ, one of at least two, specifically, one of at least three, pre-determined applicable ranges of relative humidity rH_{appl-range(n)}.

The method still further comprises:
iii) determining, specifically by the processor of the mobile device, the concentration of the analyte from a color of the reagent test region in the image, based on a color formation reaction at the reagent test region having the sample of the bodily fluid applied thereto.

In step iii), the determining the analyte concentration takes into account the applicable range of relative humidity rH_{appl-range(n)} selected in step ii).

Without narrowing the scope, this disclosure specifically may be described with respect to blood glucose measurements. It shall be noted, however, that this disclosure may also be used for other types of analytical measurements using test elements.

The term “determining a concentration of an analyte in a bodily fluid,” also referred to as an “analytical measurement,” as used herein, is a broad term and is to be given its ordinary and customary meaning to a person of ordinary skill in the art and is not to be limited to a special or customized meaning. The term specifically may refer, without limitation, to a quantitatively and/or qualitatively determination of at least one analyte in an arbitrary sample or aliquot of bodily fluid. For example, the bodily fluid may comprise one or more of blood, interstitial fluid, urine, saliva or other types of body fluids, particularly blood. The result of the determining of the concentration, as an example, may be a concentration of the analyte and/or the presence or absence of the analyte to be determined. Specifically, as an example, the analytical measurement may be a blood glucose measurement, thus the result of the analytical measurement may, for example, be a blood glucose concentration. In particular, an analytical measurement result value may be determined by the analytical measurement.

Consequently, the term “analyte concentration value,” often also referred to as “analytical measurement result value,” as used herein, is a broad term and is to be given its ordinary and customary meaning to a person of ordinary skill in the art and is not to be limited to a special or customized meaning. The term specifically may refer, without limitation, to a numerical indication of an analyte concentration in a sample.

The at least one analyte, as an example, may be or may comprise one or more specific chemical compounds and/or other parameters. As an example, one or more analytes may be determined which take part in metabolism, such as blood glucose. Additionally or alternatively, other types of analytes or parameters may be determined, e.g., a pH value.

The method, as outlined above, comprises using at least one mobile device having at least one camera. The term “mobile device” as used herein is a broad term and is to be given its ordinary and customary meaning to a person of ordinary skill in the art and is not to be limited to a special or customized meaning. The term specifically may refer, without limitation, to a mobile electronics device, more specifically to a mobile communication device such as a cell phone or smartphone. Additionally or alternatively, the mobile device may also refer to a tablet computer or another type of portable computer having at least one camera and at least one processor.

The term “camera” as used herein is a broad term and is to be given its ordinary and customary meaning to a person of ordinary skill in the art and is not to be limited to a special or customized meaning. The term specifically may refer, without limitation, to a device having at least one imaging element configured for recording or capturing spatially resolved one-dimensional, two-dimensional or even three-dimensional optical data or information. As an example, the camera may comprise at least one camera chip, such as at least one CCD chip and/or at least one CMOS chip configured for recording images. As used herein, without limitation, the term “image” specifically may relate to data recorded by using a camera, such as a plurality of electronic readings from the imaging device, such as the pixels of the camera chip.

The camera, besides the at least one camera chip or imaging chip, may comprise further elements, such as one or more optical elements, e.g., one or more lenses. As an example, the camera may be a fix-focus camera, having at least one lens which is fixedly adjusted with respect to the camera. Alternatively, however, the camera may also comprise one or more variable lenses which may be adjusted, automatically or manually. This disclosure specifically shall be applicable to cameras as usually used in mobile applications such as notebook computers, tablets or, specifically, cell phones such as smart phones. Thus, specifically, the camera may be part of a mobile device which, besides the at least one camera, comprises one or more data processing devices such as one or more data processors. Other cameras, however, are feasible.

In the method, at least one image is received, the image comprising at least a part of a reagent test region associated with one of: an optical test element and/or a color reference card. Specifically, the optical test element and/or the color reference card may be provided with the reagent test region. Moreover, if the reagent test region is provided on an optical test element, a color reference card may be adapted to be associated with said optical test element having the reagent test region. The reagent test region is adapted for application of a sample of the bodily fluid, and the reagent test region is adapted to undergo, at least partially, a color formation reaction when the sample of the bodily fluid is applied to the reagent test region. The reagent test region may also be referred to as a “test field” herein. The term “optical test element” as used herein is a broad term and is to be given its ordinary and customary meaning to a person of ordinary skill in the art and is not to be limited to a special or customized meaning. The term specifically may refer, without limitation, to an arbitrary element or device configured for performing a color-change detection reaction. The optical test element may also be referred to as test strip or test element, wherein all three terms may refer to the same element. The optical test element and/or the color reference card may particularly have a reagent test region containing at least one test chemical for detecting at least one analyte. The optical test element, as an example, may comprise at least one substrate, such as at least one carrier, with the at least one reagent test region applied thereto or integrated therein. In particular, the optical test element may further comprise one or more reference areas, such as a white field and/or a black field. Additionally or alternatively, the substrate or carrier itself may be or may comprise such a reference area. As an example, the at least one carrier may be strip-shaped, thereby rendering the test element a test strip. These test strips are generally widely in use and available. One test strip may carry a single test field or a plurality of test fields having identical or different test chemicals comprised therein. The color reference card may comprise analogous features as described herein above for the optical test strip. Particularly, the color reference card may be provided in credit card format, i.e., in the size and form of a conventional credit card made of plastic. Usually, such a card-sized color reference card exhibits a plurality of reference areas, such as a white field, a black field and/or grey fields. Additionally or alternatively, a color reference card may exhibit a plurality of reference areas having a variety of reference colors, said reference colors having colors other than white, black or grey.

As further used herein, the term “reagent test region” (also referred to as a “test field” herein) is a broad term and is to be given its ordinary and customary meaning to a person of ordinary skill in the art and is not to be limited to a special or customized meaning. The term specifically may refer, without limitation, to a coherent amount of the test chemical, such as to a field, e.g., a field of round, polygonal or rectangular shape, having one or more layers of material, with at least one layer of the test field having the test chemical comprised therein. With regard to the test chemicals comprised in optical test strips, as an example reference is made to J. Hoenes et al.: The Technology Behind Glucose Meters: Test Strips, Diabetes Technology & Therapeutics, Volume 10, Supplement 1, 2008, S-10 to S-26. Other types of test chemistry are possible and may be used for performing this disclosure.

As outlined above, the method comprises receiving at least one image of at least a part of the reagent test region having the sample of the bodily fluid applied thereto, captured by the camera of the mobile device. The term “receiving at least one image” as used herein is a broad term and is to be given its ordinary and customary meaning to a person of ordinary skill in the art and is not to be limited to a special or customized meaning. The term specifically may refer, without limitation, to one or more of imaging, image recording, image acquisition, image capturing. The term “receiving at least one image” may comprise receiving and/or capturing a single image and/or a plurality of images such as a sequence of images. For example, the receiving and/or capturing of the image may comprise recording continuously a sequence of images such as a video or a movie. The receiving and/or capturing of the at least one image may be initiated by the user action or may automatically be initiated, e.g., once the presence of the at least one object within a field of view and/or within a predetermined sector of the field of view of the camera is automatically detected. These automatic image acquisition techniques are known, e.g., in the field of automatic barcode readers, such as from automatic barcode reading apps. The receiving and/or capturing of the images may take place, as an example, by acquiring a stream or “life stream” of images with the camera, wherein one or more of the images, automatically or by user interaction such as pushing a button, are stored and used as the at least one first image or the at least one second image, respectively. The image acquisition may be supported by a processor of the mobile device, and the storing of the images may take place in a data storage device of the mobile device.

The receiving and/or capturing of the at least one image may comprise receiving and/or capturing at least one image with having the sample of the bodily fluid applied to the test strip and, further and optionally, such as before capturing the image with the sample applied to the test strip, receiving and/or capturing at least one image without having the sample of the body fluid applied to the test strip. The latter image specifically may be used for comparative purposes and may also be referred to as a “blank image” or “dry image.” The sample application generally may take place, as an example, directly or indirectly, e.g., via at least one capillary element. The at least one image received and/or captured after sample application may typically also be referred to as the “wet image,” even though the sample may have dried when the image is actually captured. The wet image typically may be received and/or taken after having waited for at least a predetermined waiting time, such as after five seconds or more, in order to allow for the detection reaction to take place. Thus, as an example, the method may comprise, between receiving and/or taking the at least one optional dry image and the at least one wet image, waiting for at least a predetermined minimum amount of time. This predetermined minimum amount of time specifically may be sufficient for a detection reaction to take place in the test strip. As an example, the minimum amount of waiting time may be at least 5 s.

The method comprises determining the analyte concentration, particularly an analyte concentration value, from color formation of the reagent test region. Thus, the method may be an analytical measurement including a change of at least one optical property of an optical test element, such as an optical test strip, which change may be measured or determined visually by using the camera. Specifically, the analytical measurement may be or may comprise a color formation reaction in the presence of the at least one analyte to be determined. The term “color formation reaction” as used herein is a broad term and is to be given its ordinary and customary meaning to a person of ordinary skill in the art and is not to be limited to a special or customized meaning. The term specifically may refer, without limitation, to a chemical, biological or physical reaction during which a color, specifically a reflectance, of at least one element involved in the reaction, changes with the progress of the reaction. The color formation may be detected by the mobile device, such as by a processor of the mobile device, and may be evaluated quantitatively, such as by deriving, from the at least one image, at least one parameter quantifying or characterizing the color formation of the test field due to the presence of the analyte in the bodily fluid. To this end, one or more specific color coordinates may be used. Thus, the mobile device and specifically the processor of the mobile device may be configured for determining a color change by determining a change of one or more color coordinates taking place due to the detection reaction.

The concentration of the analyte, particularly the analyte concentration value, is determined from the color formation of the test field. For this purpose, the at least one image is used. The analyte concentration value, as an example, may be a numerical value indicator of a result of the analytical measurement, such as indicative of the concentration of at least one analyte in the sample, such as a blood glucose concentration.

The image received in step i) further comprises at least a part of at least one hydrochromic indicator field associated with the optical test element and/or associated with the color reference card, specifically associated with the color reference card. For example, the at least one hydrochromic indicator field may be attached to the optical test element, or the at least one hydrochromic indicator field may be attached to the color reference card. Alternatively or additionally, the at least one hydrochromic indicator field may be provided separately from the optical test element and from the color reference card, but may be used together with the optical test element and/or with the color reference card. The hydrochromic indicator field exhibits at least one optically detectable color change at one or more pre-determined threshold levels of relative humidity rH_{threshold(m)}.

The relative humidity of an air-water mixture is defined as the ratio of the partial pressure of water vapor in the mixture to the equilibrium vapor pressure of water over a flat surface of pure water at a given temperature. In other words, relative humidity is the ratio of how much water vapor is in the air and how much water vapor the air could potentially contain at a given temperature. Relative humidity is normally expressed as a percentage (herein also referred to as “% rH,” i.e., percentage relative humidity); a higher percentage means that the air-water mixture is more humid. At 100% relative humidity, the air is saturated and is at its dew point. Commonly used devices for measuring humidity of air include psychrometers and hygrometers, as the skilled person is aware of. An overview of such devices can be found, e.g., in Kohlrausch, F., Praktische Physik 1, Kose, V.; Wagner, S., Hrsg., 24. Aufl.; Teubner: Stuttgart, (1996), S. 400. Any values of relative humidity, also referred to as “rH” values herein, as well as any levels of relative humidity, including threshold levels of relative humidity, e.g., rH_{threshold(m)}, rH_{threshold(m1)}, rH_{threshold(m2)}, and rHₑₛₜᵢₘₐₜₑ, used herein are provided as numerical values having the unit of “% rH,” i.e., percentage of relative humidity. Usually, any rH values referred to herein may be associated to a given ambient temperature and/or to a given ambient pressure. Generally, in the context of this disclosure, the ambient temperature may be any temperature of from 0° C. to 65° C., for example, any of 0, 5, 10, 15, 20, 25, 30, 35, 40, 45, 50, 55, 60 and 65° C., specifically any of 20 or 25° C. Furthermore, generally, in the context of this disclosure, the ambient pressure may be any pressure of from 900 hPa to 1080 hPa (mean sea-level pressure, MSLP), for example, any of 990, 1013, 1040 hPa.

In step ii), an estimate value rHₑₛₜᵢₘₐₜₑ of the local relative humidity is derived from the color of the hydrochromic indicator field in the image. The term “local” used in this context refers to the relative humidity at a current location of the mobile device, for example, an indoor location or an outdoor location. Further, the term “local” used herein may refer to any place or area which may be specified or defined in order to appropriately represent or approximate the relative humidity condition at or within a locally restricted surrounding of the mobile device. For example, it usually may be appropriate to refer to a relative humidity condition of a region (e.g., a city, a part of a city, a neighborhood, a landscape or a part thereof, a county or a federal state, etc.), particularly if the current location of the mobile device is outdoors. In this regard, the “local relative humidity” may relate to information on relative humidity as provided by any commonly available online weather service, as far as information on local relative humidity is provided. If the current location of the mobile device is indoors, alternatively or additionally, it may be appropriate to refer to a relative humidity condition within a housing or within a room.

The method may further comprise the step of displaying the analyte concentration value, such as on a display of the mobile device. Additionally or alternatively, the method may comprise storing the at least one analyte concentration value in at least one data storage device of the mobile device. Again additionally and alternatively, the method may further comprise transmitting the at least one analyte concentration value via at least one interface and/or via at least one data transmission network, such as to another computer, e.g., for further evaluation.

Accordingly, in the first aspect, this disclosure particularly relates to a computer-implemented, specifically to an in-vitro, analytical method for determining a concentration of an analyte in a bodily fluid by using a mobile device having at least one camera, and specifically having at least one processor, comprising:
i) receiving, specifically by the processor of the mobile device, at least one image captured by the camera of the mobile device, the image comprising at least a part of a reagent test region associated with an optical test element and/or associated with a color reference card, the reagent test region having a sample of the bodily fluid applied thereto, the image further comprising at least a part of at least one hydrochromic indicator field associated with the optical test element and/or associated with the color reference card, wherein the hydrochromic indicator field exhibits at least one optically detectable color change at a pre-determined threshold level of relative humidity rH_{threshold(m)}; specifically, at two or more different pre-determined threshold levels of relative humidity rH_{threshold(m)};
ii) deriving, specifically by the processor of the mobile device, from the color of the hydrochromic indicator field in the image, an estimate value rHₑₛₜᵢₘₐₜₑ of the local relative humidity; and selecting, specifically by the processor of the mobile device, based on the estimate value rHₑₛₜᵢₘₐₜₑ, one of at least two, specifically, one of at least three, pre-determined applicable ranges of relative humidity rH_{appl-range(n)}; and
iii) determining, specifically by the processor of the mobile device, the concentration of the analyte from a color of the reagent test region in the image, based on a color formation reaction at the reagent test region having the sample of the bodily fluid applied thereto, taking into account the applicable range of relative humidity rH_{appl-range(n)} selected in step ii);
   wherein each of rH_{threshold(m)} and rHₑₛₜᵢₘₐₜₑ, respectively, are rH values provided in the form of “% rH,” i.e., as percentage relative humidity.

The method proposed provides for an efficient mobile-based determination of a concentration of an analyte in a bodily fluid, by taking into account information on the relative humidity, at a current location of a mobile device, to be used for performing the method. The information on local relative humidity is obtained from hydrochromic indicator fields which are available with low effort and which are cost-efficient. Thus, a reliable accuracy of the analyte measurement can efficiently be achieved, particularly with low effort for setup and implementation.

The image received in step i) comprises at least a part of at least one hydrochromic indicator field. The hydrochromic indicator field exhibits at least one optically detectable color change at one or more, e.g., at 1, 2, 3, 4, 5, 6, 7, 8, 9 or 10, pre-determined threshold levels of relative humidity rH_{threshold(m)}; specifically, at one or two, or alternatively, at two or more, e.g., at two or three, or still alternatively, at three or more, e.g., at 3, 4, 5, 6 or 7, different pre-determined threshold levels of relative humidity rH_{threshold(m)}. Particularly, the hydrochromic indicator field may comprise one or more separate hydrochromic indicator fields, e.g., 1, 2, 3, 4, 5, 6, 7, 8, 9 or 10. Each of said separate hydrochromic indicator fields may exhibit at least one, specifically one, optically detectable color change at a pre-determined threshold level of relative humidity rH_{threshold(m)}, wherein said pre-determined threshold levels of relative humidity rH_{threshold(m)} may be partially the same, i.e., at least some redundant separate hydrochromic indicator fields may be present, or wherein said pre-determined threshold levels of relative humidity rH_{threshold(m)} may each be different from one another. For example, a hydrochromic indicator field may comprise a specific number of separate hydrochromic indicator fields, e.g., in the range of from 1 to 10, such as 3, 4, 5, 6 or 7, which each exhibit one optically detectable color change at a different pre-determined threshold level of relative humidity rH_{threshold(m)}. Thus, such a hydrochromic indicator field may comprise 7 separate hydrochromic indicator fields, said separate hydrochromic indicator fields each exhibiting an optically detectable color change at a different pre-determined threshold level of relative humidity rH_{threshold(m)}, with pre-determined threshold levels of relative humidity rH_{threshold(1)}=20% rH, rH_{threshold(2)}=30% rH, rH_{threshold(3)}=40% rH, rH_{threshold(4)}=50% rH, rH_{threshold(5)}=60% rH, rH_{threshold(6)}=70% rH, and rH_{threshold(7)}=80% rH. Providing the at least one hydrochromic indicator field in the form of a plurality of separate hydrochromic indicator fields may significantly facilitate the detection of the local relative humidity which, in step ii), is derived from the color of the hydrochromic indicator field in the image: Since the positions of the separate hydrochromic indicator fields in the image may be known or may be pre-determined, e.g., by their known location on the optical test element and/or on the color reference card, a color change occurring at any of such known or pre-determined positions can be determined more easily, and thus more accurately. Hence, from such a configuration of the at least one hydrochromic indicator field, the estimate value rHₑₛₜᵢₘₐₜₑ of the local relative humidity can be derived more reliably in step ii). However, for the purpose of this disclosure, it may also be feasible that one or more single hydrochromic indicator fields exhibit more than one color change at two or more different pre-determined threshold levels of relative humidity rH_{threshold(m)}.

Materials for the hydrochromic indicator field to be used herein may provide for a reversible or for a non-reversible color change, specifically for a reversible color change. Suitable color changing materials are commercially available and may comprise, for example, compounds selected from the list comprising cobalt dichloride, cobalt dibromide, and copper oxide. Such color changing materials may be commercially obtained, e.g., from the company Long Life for Art (Germany), see https://llfa.eu. Generally, such color changing materials are provided in the form of a test paper which is impregnated with a solution of said materials.

It is particularly useful if each of the at least one optically detectable color changes occurs, essentially completely, within at least one narrow interval of rH values, wherein each of said at least one narrow intervals of rH values is essentially centered around at least one of the pre-determined threshold levels of relative humidity rH_{threshold(m)}. Specifically, each of said at least one narrow intervals of rH values may comprise rH values spanning a range of no more than 15% rH; and more specifically, a range of no more than 10% rH; and even more specifically, a range of no more than 5% rH. In this context, the term “essentially completely” may refer to a degree of color change from a first color to a second color, the second color being visibly discernible from the first color; specifically, wherein said color change from said first color to said second color takes place at a degree of at least 80%; more specifically, wherein said degree of color change of at least 80% takes place between a degree of color change of from 10% to 90%, the degree of color change in each case being relative to the complete color change from said first color to said second color which complete color change represents a degree of 100%. Furthermore, in this context, the term “essentially centered around” may refer to a configuration wherein the pre-determined threshold level of relative humidity rH_{threshold(m)} represents a given rH value, e.g., 30% rH, and the narrow interval of rH values, e.g., a range of at most 10% rH values, is adapted such that the pre-determined threshold level of relative humidity rH_{threshold(m)} is, at least approximately, at the center of said narrow interval of rH values; thus, in this example, with a pre-determined threshold level of relative humidity rH_{threshold(m)} of 30% rH, said narrow interval of rH values covering a range of at most 10% rH values results in a range of rH values of from 25% rH to 35% rH, which is equal to 30% rH ±5% rH.

In step i), the pre-determined threshold level, or threshold levels, of relative humidity rH_{threshold(m)} may comprise one or more rH values selected from 10, 15, 20, 25, 30, 45, 50, 55, 60, 65, 70, 75 and 80% rH. For example, the hydrochromic indicator field may exhibit optically detectable color changes at the following pre-determined threshold levels of relative humidity: rH_{threshold(1)}=20% rH, rH_{threshold(2)}=30% rH, rH_{threshold(3)}=40% rH, rH_{threshold(4)}=50% rH, rH_{threshold(5)}=60% rH, rH_{threshold(6)}=70% rH, rH_{threshold(7)}=80% rH. As another example, the hydrochromic indicator field may exhibit optically detectable color changes at the following pre-determined threshold levels of relative humidity: rH_{threshold(1)}=30% rH, rH_{threshold(2)}=40% rH, rH_{threshold(3)}=50% rH.

Specifically, the at least one hydrochromic indicator field may comprise one or more separate hydrochromic indicator fields, each of which exhibits, independently from one another, at least one optically detectable color change at a, specifically different, pre-determined threshold level of relative humidity rH_{threshold(m)}, said pre-determined threshold level of relative humidity rH_{threshold(m)} in each case comprising one or more, specifically one, rH values selected from 10, 15, 20, 25, 30, 45, 50, 55, 60, 65, 70, 75 and 80% rH.

In step ii), an estimate value rHₑₛₜᵢₘₐₜₑ of the local relative humidity is derived, specifically by the processor of the mobile device, from the color of the hydrochromic indicator field in the image. Specifically, when the hydrochromic indicator field exhibits an optically detectable color change at a certain pre-determined threshold level of relative humidity rH_{threshold(m)}, e.g., at rH_{threshold(m)}=30% rH, then the estimate value rHₑₛₜᵢₘₐₜₑ of the local relative humidity derived therefrom may be, at least essentially, equal to said certain pre-determined threshold level of relative humidity rH_{threshold(m)}, such that an estimate value of the local relative humidity of rHₑₛₜᵢₘₐₜₑ=30% rH may result.

If the at least one hydrochromic indicator field comprises more than one separate hydrochromic indicator fields, e.g., 7, each of them exhibiting an optically detectable color change at a different pre-determined threshold level of relative humidity rH_{threshold(m)}, e.g., at rH threshold values equal to 20, 30, 40, 50, 60, 70, and 80% rH, then the estimate value rHₑₛₜᵢₘₐₜₑ of the local relative humidity derived therefrom may be equal to the highest one of all of those pre-determined threshold levels of relative humidity rH_{threshold(m)} which actually exhibit an optically detectable color change when the method is performed. In such a case, for the example of the afore-mentioned 7 different pre-determined threshold levels of relative humidity rH_{threshold(m)}, an estimate value of the local relative humidity of rHₑₛₜᵢₘₐₜₑ=60% rH may result, if the five pre-determined threshold levels of relative humidity rH_{threshold(1)}=20% rH, rH_{threshold(2)}=30% rH, rH_{threshold(3)}=40% rH, rH_{threshold(4)}=50% rH, and rH_{threshold(5)}=60% rH actually exhibit an optically detectable color change when the method is performed; wherein the remaining two pre-determined threshold levels of relative humidity rH_{threshold(6)}=70% rH and rH_{threshold(7)}=80% rH would not actually exhibit such an optically detectable color change when the method is performed because their respective rH threshold values rH_{threshold(m)} are higher than the actual local relative humidity.

Alternatively, other ways of deriving an estimate value rHₑₛₜᵢₘₐₜₑ of the local relative humidity, specifically by the processor of the mobile device, from the color of the hydrochromic indicator field in the image may be contemplated herein.

In step ii), furthermore, one of at least two, specifically, one of at least three, pre-determined applicable ranges of relative humidity rH_{appl-range(n)} is selected, specifically by the processor of the mobile device, based on the estimate value rHₑₛₜᵢₘₐₜₑ of the local relative humidity. Specifically, the one pre-determined applicable range of relative humidity rH_{appl-range(n)} may be selected such that it comprises the estimate value rHₑₛₜᵢₘₐₜₑ of the local relative humidity. For example, if the estimate value of the local relative humidity is rHₑₛₜᵢₘₐₜₑ=70% rH, then a pre-determined applicable range of relative humidity rH_{appl-range(n)} may be selected which comprises rH values of >60%, e.g., rH values of from >60% to ≤90% rH.

Specifically, in step ii), the at least two, specifically at least three, pre-determined applicable ranges of relative humidity rH_{appl-range(n)} are distinct from one another and do not overlap with each other. Usually, the at least two, specifically at least three, pre-determined applicable ranges of relative humidity rH_{appl-range(n)} are defined such that they form a continuous range of rH values. Such a continuous range of rH values may span a range of rH values of from 0% rH to 100% rH, e.g., of from 0% rH to 90% rH, or from 10% rH to 80% rH. Specifically, the upper threshold rH value of a pre-determined applicable range of relative humidity, e.g., rH_{appl-range(1)_low} having rH values of <30% rH and thus having an upper threshold of rH_{threshold(k)}=30% rH (with the threshold rH value of 30% rH being excluded from said first range of rH values), may itself be the lower threshold for another adjacent pre-determined applicable range of relative humidity, e.g., rH_{appl-range(2)_high} having rH values of ≥30% rH and thus having a lower threshold of rH_{threshold(k1)}=30% rH (with the threshold rH value of 30% rH being included in said second range of rH values).

If there are two pre-determined applicable ranges of relative humidity rH_{appl-range(n)}, then the first one may be a range of low relative humidity, e.g., rH_{appl-range(1)} having rH values of <30% rH, and the second one may be a range of high relative humidity, e.g., rH_{appl-range(2)} having rH values of >60% rH. Generally, the at least two pre-determined applicable ranges of relative humidity rH_{appl-range(n)} may comprise a first applicable range of relative humidity rH_{appl-range(1)_low}, having rH values X1_{rH} of 0<X1_{rH}<rH_{threshold(k)}, and a second applicable range of relative humidity rH_{appl-range(2)_high}, having rH values X2_{rH} of rH_{threshold(k)}≤X2_{rH}.

Alternatively, the at least two pre-determined applicable ranges of relative humidity rH_{appl-range(n)} may comprise at least three pre-determined applicable ranges of relative humidity rH_{appl-range(n)}. Specifically, the at least three pre-determined applicable ranges of relative humidity rH_{appl-range(n)} may comprise at least a first applicable range of relative humidity rH_{appl-range(1)_low}, having rH values X1_{rH} of 0<X1_{rH}<rH_{threshold(k1)}, and a second applicable range of relative humidity rH_{appl-range(2)_medium}, having rH values X2_{rH} of rH_{threshold(k1)}≤X2_{rH}≤rH_{threshold(k2)}, and a third applicable range of relative humidity rH_{appl-range(3)_high}, having rH values X3_{rH} of rH_{threshold(k2)}<X3_{rH}.

With regard to the foregoing exemplary rH ranges, particularly, rH_{threshold(k)} may be an rH value selected from one of 15, 30, 45, 60 and 75% rH; or rH_{threshold(k1)} may be an rH value selected from one of 15, 20, 25, 30, 35, and 40% rH, and rH_{threshold(k2)} may be an rH value selected from one of 50, 55, 60, 65, 70, and 75% rH. Specifically, rH_{threshold(k)} may be an rH value selected from one of 30 and 60% rH. Alternatively, rH_{threshold(k1)} may be an rH value selected from one of 25, 30, and 35% rH, and rH_{threshold(k2)} may be an rH value selected from one of 55, 60, and 65% rH.

Herein, each of X1_{rH}, X2_{rH}, X3_{rH}, rH_{threshold(m)}, rH_{threshold(k)}, rH_{threshold(k1)}, and rH_{threshold(k2)}, respectively, are rH values provided in the unit of “% rH,” specifically at a given ambient temperature and/or at a given ambient pressure.

In step iii), the concentration of the analyte in the bodily fluid is determined, specifically by the processor of the mobile device, from a color of the reagent test region in the image, based on a color formation reaction at the reagent test region having the sample of the bodily fluid applied thereto. The determining the concentration of the analyte takes into account the applicable range of relative humidity rH_{appl-range(n)} selected in step ii).

Advantageously, the determining the concentration of the analyte in step iii) may comprise determining, specifically by the processor of the mobile device, at least one relative humidity correction rH_{corr} and/or at least one relative humidity correction function rH_{corr-fct}, independently from one another, in each case based on one of the applicable ranges of relative humidity rH_{appl-range(n)} selected in step ii). Said at least one relative humidity correction rH_{corr} and/or said at least one relative humidity correction function rH_{corr-fct} may be taken into account in step iii) for the determining of the concentration of the analyte.

The term “relative humidity correction rH_{corr}” as used herein is a broad term and is to be given its ordinary and customary meaning to a person of ordinary skill in the art and is not to be limited to a special or customized meaning. The term specifically may refer, without limitation, to one or more of a specified relative humidity value, an average relative humidity value, a representative relative humidity value, a reference relative humidity value, a range of relative humidity values, and a delta relative humidity value, particularly a delta relative humidity value from a reference relative humidity value. Additionally or alternatively, the term “relative humidity correction rH_{corr}” may refer, without limitation, to one or more of a delta analyte concentration value, an average delta analyte concentration value, a representative delta analyte concentration value, a reference delta analyte concentration value, a range of delta analyte concentration values; specifically, a delta analyte concentration value. The term “relative humidity correction function rH_{corr-fct}” may refer to a mathematical function, factor, formula, or algorithm, each of which may be applied in the determination of the analyte concentration in step iii). Specifically, the term “relative humidity correction function _{rHcorr-fct}” may comprise using, without limitation, one or more of a specified relative humidity value, an average relative humidity value, a representative relative humidity value, a reference relative humidity value, a range of relative humidity values, a delta relative humidity value, a delta analyte concentration value, an average delta analyte concentration value, a representative delta analyte concentration value, a reference delta analyte concentration value, and a range of delta analyte concentration values. For practical implementation, the “relative humidity correction rH_{corr}” and/or the “relative humidity correction function rH_{corr-fct}” advantageously comprise a delta analyte concentration value. The pre-determined applicable range of relative humidity rH_{appl-range(n)} selected in step ii), derived from the estimate value rHₑₛₜᵢₘₐₜₑ of the local relative humidity, may be used as an input, or as a trigger for applying said function, factor, formula, or algorithm, in the determination of the analyte concentration in step iii). Generally, the “relative humidity correction rH_{corr}” and the “relative humidity correction function rH_{corr-fct}” may be useful to appropriately take into account any impact of relative humidity on chemical reactions as used herein in a reagent test region of an optical test element. Such impacts may, e.g., be determined empirically by a skilled person for any specific type of chemical test reagent. In some cases, the determining of the analyte concentration in step iii) based on the color formation reaction at the reagent test region may be assumed to take place at typical relative humidities, such as at an ambient relative humidity of, e.g., about 45% rH. In such cases, any influence of relative humidity on the chemical reaction may not explicitly be represented by a factor, a formula, or an algorithm using the local relative humidity as an input; in these cases, the “relative humidity correction rH_{corr}” may be determined to be set as an ambient relative humidity, such as 45% rH. Alternatively, in these cases the “relative humidity correction function rH_{corr-fct}” may be determined to be a multiplication factor equal to “1,” i.e., not affecting the calculation of the analyte concentration.

One or more, optionally each, of the applicable ranges of relative humidity rH_{appl-range(n)} may be associated with their own relative humidity correction rH_{corr} and/or relative humidity correction function rH_{corr-fct}. Specifically, said relative humidity corrections rH_{corr} and/or relative humidity correction functions rH_{corr-fct} may be selected independently from one another for each applicable range of relative humidity rH_{appl-range(n)}.

Particularly, it may be contemplated that at least one of the applicable ranges of relative humidity rH_{appl-range(n)} is not associated with a relative humidity correction rH_{corr} and/or relative humidity correction function rH_{corr-fct}, such that, for said at least one of the applicable ranges of relative humidity rH_{appl-range(n)}, no correction for relative humidity is taken into account for the determining of the concentration of the analyte. For example, a relative humidity correction rH_{corr} and/or relative humidity correction function rH_{corr-fct} may be associated with an applicable range of relative humidity rH_{appl-range(n)} which has high rH values, such as rH_{appl-range(2)_high} having rH values X2_{rH} of rH_{threshold(k)}≤X2_{rH}, e.g., with rH_{threshold(k)}=30% rH or with rH_{threshold(k)}=60% rH, whereas another applicable range of relative humidity rH_{appl-range(n)} which has low rH values, such as rH_{appl-range(1)_low} having rH values X1_{rH} of 0<X1_{rH}<rH_{threshold(k)}, e.g., with rH_{threshold(k)}=30% rH or with rH_{threshold(k)}=60% rH, is not associated with a relative humidity correction rH_{corr} and/or relative humidity correction function rH_{corr-fct}. Additionally or alternatively, one or more of the relative humidity corrections rH_{corr} and/or relative humidity correction functions rH_{corr-fct} may be provided in a form, e.g., in the form of a number, a factor, a parameter, and/or a function, such that effectively, for said applicable ranges of relative humidity rH_{appl-range(n)}, no correction for relative humidity is applied when said relative humidity correction rH_{corr} and/or said relative humidity correction function rH_{corr-fct} is taken into account for the determining of the concentration of the analyte in step iii).

Specifically, in step iii), the taking into account said at least one relative humidity correction rH_{corr} and/or said at least one relative humidity correction function rH_{corr-fct} further may comprise associating one or more, specifically one, two or three, more specifically each, of said relative humidity corrections rH_{corr} and/or of said relative humidity correction functions rH_{corr-fct}, independently from one another, with a pre-defined concentration range of the analyte concentration. Said pre-defined concentration range may be selected from at least two, specifically from at least three, pre-defined analyte concentration ranges. Specifically, the pre-defined analyte concentration ranges are distinct from one another and do not overlap with each other.

In this regard, similar as it is usually the case for the pre-determined applicable ranges of relative humidity rH_{appl-range(n)} described herein above, usually the at least two, specifically at least three, pre-defined analyte concentration ranges are defined such that they form a continuous range of analyte concentration values. Such a continuous range of analyte concentration values may span a full range of analyte concentration values which may be obtained for any specific analyte in a bodily fluid. Specifically, similar as it has been described herein above for the pre-determined applicable ranges of relative humidity rH_{appl-range(n)}, the upper threshold analyte concentration value of a pre-defined analyte concentration range (which pre-defined analyte concentration range, e.g., may not comprise the threshold concentration value) may itself be the lower threshold analyte concentration value for another adjacent pre-defined analyte concentration range (which pre-defined analyte concentration range, e.g., may comprise the threshold concentration value).

Specifically, it may be contemplated that at least one of the pre-defined concentration ranges of the analyte concentration is not associated to a relative humidity correction rH_{corr} and/or relative humidity correction function rH_{corr-fct}, such that, for said at least one of the pre-defined concentration ranges of the analyte concentration, no correction for relative humidity is taken into account for the determining of the concentration of the analyte in step iii). Additionally or alternatively, for at least one of the pre-defined concentration ranges of the analyte concentration, the relative humidity correction rH_{corr} and/or the relative humidity correction function rH_{corr-fct} associated thereto, may be provided in a form, e.g., a number, a factor, a parameter, and/or a function, such that, for said at least one of the pre-defined concentration ranges of the analyte concentration, no correction for relative humidity is taken into account for the determining of the concentration of the analyte in step iii).

In a particular aspect, the method described herein above may further comprise receiving, specifically by the mobile device, at least one additional estimate value rH_{add-estimate} of the local relative humidity from at least one of:
a) a remote weather service, via a wireless connection to the mobile device;
b) an external electronic device, comprising an ambient humidity sensor, via a wireless connection to the mobile device; and
c) an ambient humidity sensor located in the mobile device.

Furthermore, step ii) may comprise verifying or adjusting, specifically by the processor of the mobile device, the estimate value _{rHestimate} of the local relative humidity, by taking into account at least one of the additional estimate values rH_{add-estimate}.

Herein, each of rHₑₛₜᵢₘₐₜₑ and rH_{add-estimate}, respectively, are rH values provided in the form of “% rH,” specifically at a given ambient temperature and/or at a given ambient pressure.

Specifically, the external electronic device may be selected from one or more of wearables, such as fitness trackers, smart watches, smart glasses, smart clothing; smart-home components, such as electronic heating systems, smart temperature measurement units, home weather stations; and body-worn sensors, such as non-invasive analyte measurement sensors, provided that the respective external electronic device comprises an ambient humidity sensor and a capability to wirelessly connect to the mobile device, e.g., via WiFi, Bluetooth, BLE, or the like.

In another aspect, this disclosure relates to a mobile device having at least one camera and at least one processor, the mobile device being configured for determining a concentration of an analyte in a bodily fluid, wherein the at least one concentration of the analyte is determined from a color formation reaction at a reagent test region; and wherein the mobile device further is configured for performing at least steps i) to iii) of the computer-implemented analytical method, specifically of the computer-implemented in-vitro analytical method, described herein.

In another aspect, this disclosure relates to a kit, comprising:
a mobile device having at least one camera and at least one processor, the mobile device being configured for determining a concentration of an analyte in a bodily fluid, wherein the at least one concentration of the analyte is determined from a color formation reaction at a reagent test region; and wherein the mobile device further is configured for performing at least steps i) to iii) of the computer-implemented analytical method, specifically of the computer-implemented in-vitro analytical method, described herein; and
at least one of an optical test element and a color reference card, said optical test element and/or said color reference card being associated with the reagent test region.

In another aspect, this disclosure relates to a computer program comprising instructions which, when the program is executed by:
a mobile device having at least one camera and at least one processor, the mobile device being configured for determining a concentration of an analyte in a bodily fluid, wherein the at least one concentration of the analyte is determined from a color formation reaction at a reagent test region; and wherein the mobile device further is configured for performing at least steps i) to iii) of the computer-implemented analytical method, specifically of the computer-implemented in-vitro analytical method, described herein;
   cause the mobile device to carry out at least steps i) to iii) of the computer-implemented analytical method, specifically of the computer-implemented in-vitro analytical method, described herein.

In another aspect, this disclosure relates to a computer-readable storage medium comprising instructions which, when executed by:
a mobile device having at least one camera and at least one processor, the mobile device being configured for determining a concentration of an analyte in a bodily fluid, wherein the at least one concentration of the analyte is determined from a color formation reaction at a reagent test region; and wherein the mobile device further is configured for performing at least steps i) to iii) of the computer-implemented analytical method, specifically of the computer-implemented in-vitro analytical method, described herein;
   cause the mobile device to carry out at least steps i) to iii) of the computer-implemented analytical method, specifically of the computer-implemented in-vitro analytical method, described herein.

### BRIEF DESCRIPTION OF THE DRAWINGS

The above-mentioned aspects of exemplary embodiments will become more apparent and will be better understood by reference to the following description of the embodiments taken in conjunction with the accompanying drawings, wherein:

FIG. 1 shows the impact of humidity on blood glucose measurement values;

FIG. 2 shows an example of several relative humidity correction functions rH_{corr-fct};

FIG. 3 shows an embodiment of a kit and a mobile device for performing an analytical measurement, illustrated in a perspective view; and

FIG. 4 illustrates a flow chart of an exemplary embodiment for carrying out the method of this disclosure.

### DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

Herein, all rH values mentioned are provided in the form of “% rH,” i.e., percentage relative humidity, including, e.g., rH_{threshold(m)}, rH_{threshold(k)} rHₑₛₜᵢₘₐₜₑ, and the like.

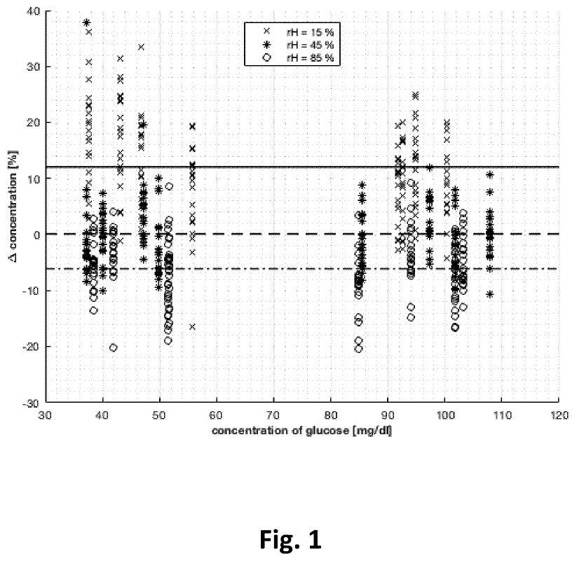

FIG. 1 shows the impact of humidity on blood glucose measurement values in a range of analyte concentration which is below 120 mg/dl, without applying a correction for relative humidity, measured by the Accu-Chek® SugarView® app of Roche Diabetes Care GmbH (Germany), using Accu-Chek® Active optical test strips from Roche Diabetes Care GmbH (Germany). At higher analyte concentrations of about 450 mg/dl (not illustrated) no significant dependence on relative humidity can be observed.

In FIG. 1, deviations of the measurement values, which deviations can be attributed to an impact of relative humidity, are represented as a delta concentration percentage, relative to the average measurement value at 45% rH. The indicated lines represent the averages of the deviations at 15% rH, at 45% rH, and at 85% rH, respectively.

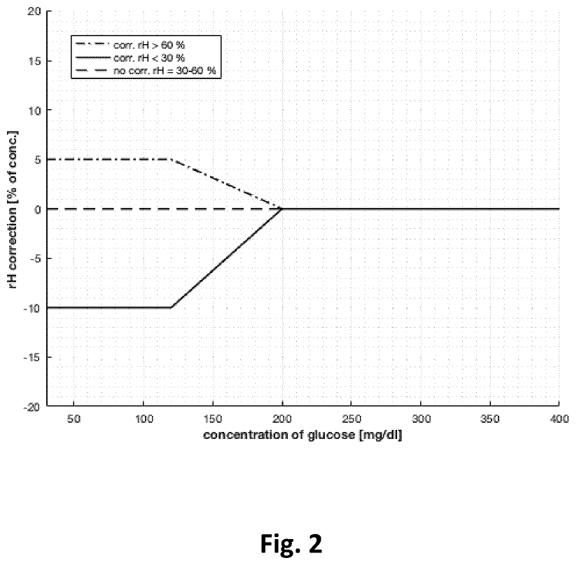

FIG. 2 shows an example of several relative humidity correction functions rH_{corr-fct}, each of which is associated with one of a plurality of applicable ranges of relative humidity rH_{appl-range(n)}. In this example, each relative humidity correction function rH_{corr-fct} is associated with one of three applicable ranges of relative humidity rH_{appl-range(n)}. Specifically, a first relative humidity correction functions rH_{corr-fct(1)} is associated with a first applicable range of relative humidity rH_{appl-range(1)_low}, having rH values X1_{rH} of 0<X1_{rH}<rH_{threshold(k1)}, wherein rH_{threshold(k1)}=30% rH; and a second relative humidity correction functions rH_{corr-fct(2)} is associated with a second applicable range of relative humidity rH_{appl-range(2)_medium}, having rH values X2_{rH} of rH_{threshold(k1)}≤X2_{rH}≤rH_{threshold(k2)}, wherein rH_{threshold(k1)}=30% rH and wherein rH_{threshold(k2)}=60% rH; and a third relative humidity correction functions rH_{corr-fct(3)} is associated with a third applicable range of relative humidity rH_{appl-range(3)_high}, having rH values X3_{rH} of rH_{threshold(k2)}<X3_{rH}, wherein rH_{threshold(k2)}=60% rH.

Furthermore, in FIG. 2, each of the three relative humidity correction functions rH_{corr-fct(1)}, rH_{corr-fct(2)} and rH_{corr-fct(3)} is associated with three pre-defined concentration ranges of the analyte concentration. Specifically, a first pre-defined concentration range of the analyte concentration has concentration values of from 0 mg/dl to <120 mg/dl; a second pre-defined concentration range of the analyte concentration has concentration values of from 120 mg/dl to 200 mg/dl; and a third pre-defined concentration range of the analyte concentration has concentration values of >200 mg/dl. Thus, if a measurement value is measured in the first analyte concentration range below 120 mg/dl, then a constant correction is applied for the determining the concentration of the analyte in step iii), for each of the three applicable ranges of relative humidity rH_{appl-range(n)} which each has its own relative humidity correction function rH_{corr-fct}. In the second analyte concentration range (120 mg/dl-200 mg/dl), for two of the three applicable ranges of relative humidity rH_{appl-range(n)}, the correction factor applied to the measurement value is linearly reduced until a correction factor of 1 results, such that no correction for relative humidity is taken into account anymore. In the third analyte concentration range of >200 mg/dl, no correction for relative humidity is applied or taken into account, for each of the three applicable ranges of relative humidity rH_{appl-range(n)}.

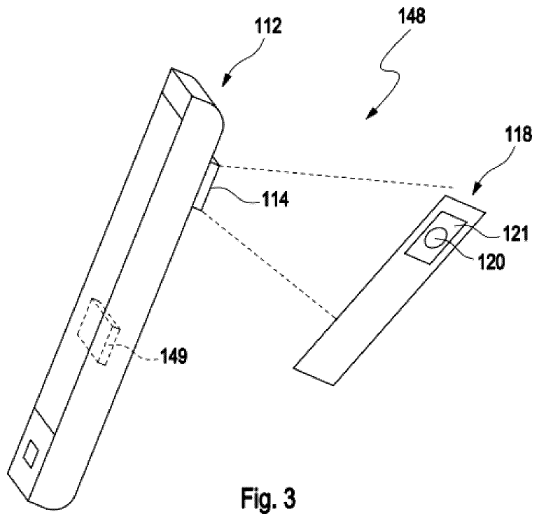

In FIG. 3, an embodiment of a kit 148 and a mobile device for performing an analytical measurement is illustrated in a perspective view. The kit 148 comprises the at least one mobile device 112 and the at least one optical test element 118, namely a test strip configured for performing a color-change detection reaction. The mobile device 112, having a camera 114, may further comprise a processor 149. The mobile device 112, specifically by using the processor 149, may be configured for performing the method described herein. The optical test element 118 is an optical test strip. In particular, the optical test strip 118 may specifically have at least one reagent test region 120, the reagent test region 120 containing at least one test chemical for detecting at least one analyte in the sample. The optical test strip 118 further contains a hydrochromic indicator field 121. The mobile device 112, as illustrated in FIG. 3, may capture the at least one image 124 of at least a part of the reagent test region 120 associated with the optical test strip 118, by using the camera 114.

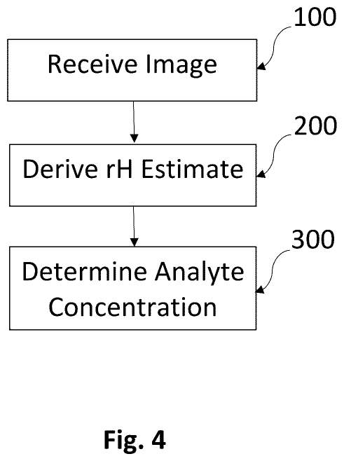

FIG. 4 illustrates a flow chart of an exemplary embodiment for carrying out the method of this disclosure, making use of the kit as shown in FIG. 3.

The computer-implemented in-vitro analytical method for determining a concentration of an analyte in a bodily fluid by using the mobile device 112 having at least one camera 114 and least one processor 149, comprises, in a first step i), depicted with reference numeral 100 in FIG. 4, receiving, specifically by the processor 149 of the mobile device 112, the at least one image 124 captured by the camera 114 of the mobile device 112. Herein, the image 124 comprises at least a part of the reagent test region 120 associated with the optical test element 118. Alternatively, the image 124 comprising at least a part of the reagent test region 120 may be associated with a color reference card 118. The reagent test region 120 has a sample of the bodily fluid applied thereto.

Furthermore, the image 124 comprises at least a part of at least one hydrochromic indicator field 121 associated with the optical test element 118. Alternatively, the hydrochromic indicator field 121 may be associated with a color reference card 118. The hydrochromic indicator field 121 exhibits at least one optically detectable color change at a pre-determined threshold level of relative humidity rH_{threshold(m)}. For example, the hydrochromic indicator field 121 may exhibit an optically detectable color change at two or more different pre-determined threshold levels of relative humidity rH_{threshold(m)}. Advantageously, the at least one hydrochromic indicator field 121 comprises a plurality of separate hydrochromic indicator fields, each of which exhibits an optically detectable color change at a different pre-determined threshold level of relative humidity rH_{threshold(m)}. The pre-determined threshold levels of relative humidity rH_{threshold(m)} may, in each case, i.e., for each of the separate hydrochromic indicator fields, comprise an rH value selected from 10, 15, 20, 25, 30, 45, 50, 55, 60, 65, 70, 75 and 80% rH, e.g., the following 7 different rH values may be selected as the pre-determined threshold levels of relative humidity rH_{threshold(1)} to rH_{threshold(7)}: 20, 30, 40, 50, 60, 70, and 80% rH.

Generally, each of the at least one optically detectable color changes occurs, essentially completely, within a narrow interval of rH values. Each of said narrow intervals of rH values may essentially be centered around one of the pre-determined threshold levels of relative humidity rH_{threshold(m)}. Advantageously, each of the narrow intervals of rH values comprises rH values spanning a range of no more than 15% rH.

The exemplary embodiment illustrated in FIG. 4 for carrying out the method further comprises, in a second step ii), depicted with reference numeral 200 in FIG. 4, deriving, specifically by the processor 149 of the mobile device 112, from the color of the hydrochromic indicator field 121 in the image 124, an estimate value rHₑₛₜᵢₘₐₜₑ of the local relative humidity. In the example of the 7 separate hydrochromic indicator fields, each of which exhibits an optically detectable color change at one of the different pre-determined threshold levels of relative humidity rH_{threshold(1)} to rH_{threshold(7)} (having rH values of 20, 30, 40, 50, 60, 70, and 80% rH, respectively), the estimate value rHₑₛₜᵢₘₐₜₑ of the local relative humidity may be equal to the highest one of all of those pre-determined threshold levels of relative humidity rH_{threshold(m)} which actually exhibit an optically detectable color change when the method is performed, whereas for the other remaining pre-determined threshold levels of relative humidity rH_{threshold(m)} (having rH threshold values higher than the actual local humidity) no optically detectable color change is actually observed when the method is performed. Thus, if in this example, when the method is performed an optically detectable color change is actually observed only for those two of the 7 separate hydrochromic indicator fields having a pre-determined threshold level of relative humidity rH_{threshold(1)}=20% rH and rH_{threshold(2)}=30% rH, then an estimate value of the local relative humidity of rHₑₛₜᵢₘₐₜₑ=30% rH may be derived in step ii).

Step ii) (reference numeral 200) furthermore comprises selecting, specifically by the processor 149 of the mobile device 112, based on the estimate value _{rHestimate}, one of at least two pre-determined applicable ranges of relative humidity rH_{appl-range(n)}. For example, one of the at least three pre-determined applicable ranges of relative humidity rH_{appl-range(n)} illustrated in FIG. 2 may be selected, i.e., one of the first applicable range of relative humidity rH_{appl-range(1)_low} (having rH values X1_{rH} of 0<X1_{rH}<rH_{threshold(k1)}, wherein rH_{threshold(k1)}=30% rH), the second applicable range of relative humidity rH_{appl-range(2)_medium} (having rH values X2_{rH} of rH_{threshold(k1)}≤X2_{rH}≤rH_{threshold(k2)}, wherein rH_{threshold(k1)}=30% rH and wherein rH_{threshold(k2)}=60% rH), and the third applicable range of relative humidity rH_{appl-range(3)_high} (having rH values X3_{rH} of rH_{threshold(k2)}<X3_{rH}, wherein rH_{threshold(k2)}=60% rH). Thus, based on an estimate value of the local relative humidity of rHₑₛₜᵢₘₐₜₑ=30% rH derived in the example herein above, the second applicable range of relative humidity rH_{appl-range(2)_medium} (having rH values X2_{rH} of rH_{threshold(k1)}≤X2_{rH}≤rH_{threshold(k2)}, wherein rH_{threshold(k1)}=30% rH and wherein rH_{threshold(k2)}=60% rH) is selected.

The exemplary embodiment illustrated in FIG. 4 for carrying out the method further comprises, in a third step iii), depicted with reference numeral 300 in FIG. 4, determining, specifically by the processor 149 of the mobile device 112, the concentration of the analyte from a color of the reagent test region 120 in the image, based on a color formation reaction at the reagent test region 120 having the sample of the bodily fluid applied thereto. Herein the applicable range of relative humidity rH_{appl-range(n)} selected in step ii) is taken into account, e.g., the second applicable range of relative humidity rH_{appl-range(2)_medium} herein above. As an example, the second applicable range of relative humidity rH_{appl-range(2)_medium}, having rH values of from 30% rH to 60% rH, may be assumed to represent a range of moderate relative humidity values, i.e., neither including rH values which are particularly low (e.g., <30% rH) nor including rH values which are particularly high (e.g., >60% rH); such moderate relative humidity values may have only a comparatively limited impact on the measurement values of the analyte concentration in the bodily fluid. This is, e.g., exemplified in FIG. 1, wherein the medium range of rH values (30 to 60% rH) represents a reference both for ranges of rH values having rather low rH values (<30% rH) and for ranges of rH values having rather high rH values (>30% rH), and wherein it is illustrated that for both of the rH ranges having low and high rH values, respectively, significant deviations of the measured blood glucose concentration occur upon an impact of rather low rH values (such as 15% rH) or of rather high rH values (such as 85% rH). Thus, when taking the applicable range of relative humidity rH_{appl-range(2)_medium} selected in step ii) into account when determining the concentration of the analyte from a color of the reagent test region 120 in the image in step iii), it may be appropriate not to apply a correction for relative humidity. This situation is illustrated in FIG. 2 by the dashed line which represents the applicable range of relative humidity rH_{appl-range(2)_medium}.

On the other hand, a correction for an impact of relative humidity may be appropriate, if a measurement of blood glucose concentrations is performed at rather low rH values (such as 15% rH) or at rather high rH values (such as 85% rH); deviations of the measured blood glucose concentration which occur upon an impact of such low or high rH values, respectively, are illustrated in FIG. 1 by the crosses (“x,” for 15% rH) and by the plus signs (“+,” for 85% rH). Accordingly, for the purpose of giving an example, if another one of the three pre-determined applicable ranges of relative humidity rH_{appl-range(n)} illustrated in FIG. 2 is selected (i.e., another one than the medium range), taking a correction for humidity into account may improve the accuracy of the resulting blood glucose measurement value.

To this end, the determining the concentration of the analyte in step iii) may comprise determining at least one relative humidity correction rH_{corr} and/or at least one relative humidity correction function rH_{corr-fct}, in each case based on one of the applicable ranges of relative humidity rH_{appl-range(n)} selected in step ii), and taking them into account for the determining of the concentration of the analyte in step iii). By way of example, the applicable ranges of relative humidity rH_{appl-range(n)} may each, independently from one another, be associated with their own relative humidity correction rH_{corr} and/or relative humidity correction function rH_{corr-fct}. In FIG. 2, each of the three applicable ranges of relative humidity (rH_{appl-range(1)_low}, having rH values X1_{rH} of 0<X1_{rH}<rH_{threshold(k1)}, wherein rH_{threshold(k1)}=30% rH); _{rHappl-range(2)_medium}, having rH values _{X2rH}^{of} rH_{threshold(k1)}≤X2_{rH}≤rH_{threshold(k2)}, wherein rH_{threshold(k1)}=30% rH and rH_{threshold(k2)}=60% rH; and rH_{appl-range(3)_high}, having rH values X3_{rH} of rH_{threshold(k2)}<X3_{rH}, wherein rH_{threshold(k2)}=60% rH) is associated with its own relative humidity correction function _{rHcorr-fct}. However, alternatively, some of the applicable ranges of relative humidity rH_{appl-range(n)} may not be associated with a relative humidity correction rH_{corr} and/or relative humidity correction function rH_{corr-fct}, such that, for some of the applicable ranges of relative humidity rH_{appl-range(n)}, no correction for relative humidity is taken into account for the determining of the concentration of the analyte. The latter situation may, e.g., be conceived for the second applicable range of relative humidity rH_{appl-range(2)_medium}, which was given as an example herein above.

The taking into account the relative humidity corrections rH_{corr} and/or the relative humidity correction functions rH_{corr-fct} in step iii) may further comprise associating rH_{corr} and/or rH_{corr-fct} with a pre-defined concentration range of the analyte concentration. The pre-defined concentration range is selected from at least two pre-defined analyte concentration ranges. As an example, with regard to FIG. 2, the first pre-defined concentration range of the analyte concentration has concentration values of from 0 mg/dl to <120 mg/dl; the second one has concentration values of from 120 mg/dl to 200 mg/dl; and the third one has concentration values of >200 mg/dl. In the present example, the relative humidity correction functions rH_{corr-fct} are each associated with a different one of said three pre-defined concentration ranges of the analyte concentration. In this scenario, according to FIG. 2, a different correction for relative humidity is taken into account for each of the three concentration ranges. However, alternatively, some of the pre-defined concentration ranges of the analyte concentration may not be associated to a relative humidity correction function rH_{corr-fct}, such that, for some of the pre-defined concentration ranges of the analyte concentration, no correction for relative humidity is taken into account for the determining of the concentration of the analyte in step iii). The latter situation may, e.g., be conceived for the third pre-defined concentration range of the analyte concentration, having analyte concentration values of >200 mg/dl, which was given as an example herein above.

As a specific example, an estimate value of the local relative humidity of rHₑₛₜᵢₘₐₜₑ=20% rH may be derived from the color of the hydrochromic indicator field in the image in step ii). Based on said estimate value of the local relative humidity of rHₑₛₜᵢₘₐₜₑ=20% rH, the first applicable range of relative humidity _{rHappl-range(1)_low} (having rH values X1_{rH} of 0<X1_{rH}<rH_{threshold(k1)}, wherein rH_{threshold(k1)}=30% rH) may be selected from the three pre-determined applicable ranges of relative humidity rH_{appl-range(n)}, which are illustrated in FIG. 2.

The first applicable range of relative humidity rH_{appl-range(1)_low} is further associated with the three pre-defined concentration ranges of the analyte concentration illustrated in FIG. 2, having concentration values of from 0 mg/dl to <120 mg/dl; of from 120 mg/dl to 200 mg/dl; and of >200 mg/dl, respectively. In this example, the relative humidity correction functions rH_{corr-fct}, for each of the three pre-determined applicable ranges of relative humidity rH_{appl-range(n)}, including rH_{appl-range(1)_low}, are each associated with the three pre-defined concentration ranges of the analyte concentration, such that a different correction for relative humidity is taken into account for each of the three concentration ranges.

For the first pre-determined applicable range of relative humidity rH_{appl-range(1)_low} considered in this example, and with regard to FIG. 2, a constant correction is applied for the determining the concentration of the analyte in step iii), if the measured blood glucose concentration value is below 120 mg/dl. This constant correction compensates, at least partially, for the positive deviations of the blood glucose measurement values which are observed at a relative humidity of 15% rH, and which, on average, amount to more than 10%, in terms of Δ concentration, at analyte concentration values of <120 mg/dl. For measured analyte concentrations between 120 mg/dl and 200 mg/dl, likewise, a correction is applied for the determining the concentration of the analyte in step iii), but in this case the correction linearly decreases from 120 mg/dl to 200 mg/dl. Above 200 mg/dl of measured analyte concentrations, no correction for relative humidity is applied or taken into account, respectively.

Similarly, the third pre-determined applicable range of relative humidity rH_{appl-range(3)_high} (having rH values X3_{rH} of rH_{threshold(k2)}<X3_{rH}, wherein rH_{threshold(k2)}=60% rH), which is associated with its own relative humidity correction function _{rHcorr-fct}, which itself again is further associated with the three pre-defined concentration ranges of the analyte concentration illustrated in FIG. 2, may be considered. For rH_{appl-range(3)_high}, and with regard to FIG. 2, similarly, a constant correction is applied for the determining the concentration of the analyte in step iii), if the measured blood glucose concentration value is below 120 mg/dl. However, for rH_{appl-range(3)_high}, the constant correction compensates, at least partially, for the negative deviations of the blood glucose measurement values which are observed at a relative humidity of 85% rH, and which, on average, amount to more than 5%, in terms of Δ concentration, at analyte concentration values of <120 mg/dl. For measured analyte concentrations between 120 mg/dl and 200 mg/dl, likewise, a correction is applied for the determining the concentration of the analyte in step iii), wherein the correction linearly decreases from 120 mg/dl to 200 mg/dl. Above 200 mg/dl of measured analyte concentrations, no correction for relative humidity is applied or taken into account, respectively.

While exemplary embodiments have been disclosed hereinabove, the present invention is not limited to the disclosed embodiments. Instead, this application is intended to cover any variations, uses, or adaptations of this disclosure using its general principles. Further, this application is intended to cover such departures from the present disclosure as come within known or customary practice in the art to which this invention pertains and which fall within the limits of the appended claims.

### List of Reference Numbers

112 mobile device
114 camera
118 optical test element (optical test strip; color reference card)
120 reagent test region
121 hydrochromic indicator field
124 image
148 kit
149 processor

What is claimed is:

1. An analytical method for determining a concentration of an analyte in a bodily fluid by using a mobile device having a camera, the method comprising:
i) receiving an image captured by the camera of the mobile device, the image comprising at least a part of a reagent test region associated with an optical test element and/or associated with a color reference card, the reagent test region having a sample of the bodily fluid applied thereto, the image further comprising at least a part of a hydrochromic indicator field associated with the optical test element and/or associated with the color reference card, wherein the hydrochromic indicator field exhibits an optically detectable color change at a pre-determined threshold level of relative humidity rH_{threshold(m)};
ii) deriving, from the color of the hydrochromic indicator field in the image, an estimate value rHₑₛₜᵢₘₐₜₑ of the local relative humidity, and selecting, based on the estimate value rHₑₛₜᵢₘₐₜₑ, one of at least two pre-determined applicable ranges of relative humidity rH_{appl-range(n)}; and
iii) determining the concentration of the analyte from the color of the reagent test region in the image, based on a color formation reaction at the reagent test region having the sample of the bodily fluid applied thereto, taking into account the applicable range of relative humidity rH_{appl-range(n)} selected in step ii);
wherein each of rH_{threshold(m)} and rHₑₛₜᵢₘₐₜₑ, respectively, are rH values provided in the form of % rH.

2. The method according to claim 1, wherein the optically detectable color change occurs substantially completely within a narrow interval of rH values centered around at least one of the pre-determined threshold levels and spanning a range of no more than 15% rH.

3. The method according to claim 1, wherein the hydrochromic indicator field comprises one or more separate hydrochromic indicator fields, each of which exhibits, independently from one another, at least one optically detectable color change at a different pre-determined threshold level of relative humidity rH_{threshold(m)}, said pre-determined threshold level of relative humidity rH_{threshold(m)} in each case comprising one or more rH values selected from 10, 15, 20, 25, 30, 45, 50, 55, 60, 65, 70, 75 and 80% rH.

4. The method according to claim 1, wherein the at least two pre-determined applicable ranges of relative humidity rH_{appl-range(n)} comprise:
a first applicable range of relative humidity rH_{appl-range(1)_low}, having rH values X1_{rH} of 0<X1_{rH}<rH_{threshold(k)}, and a second applicable range of relative humidity rH_{appl-range(2)_high}, having rH values X2_{rH} of rH_{threshold(k)}≤X2_{rH}; or
at least three pre-determined applicable ranges of relative humidity rH_{appl-range(n)}; specifically, wherein the at least three pre-determined applicable ranges of relative humidity rH_{appl-range(n)} comprise at least a first applicable range of relative humidity rH_{appl-range(1)_low}, having rH values _{X1rH} of 0<X1_{rH}<rH_{threshold(k1)}, and a second applicable range of relative humidity rH_{appl-range(2)_medium}, having rH values X2_{rH} of rH_{threshold(k1)}≤X2_{rH}≤rH_{threshold(k2)}, and a third applicable range of relative humidity rH_{appl-range(3)_high}, having rH values X3_{rH} of rH_{threshold(k2)}<X3_{rH};
wherein each of X1_{rH}, X2_{rH}, X3_{rH}, rH_{threshold(k)}, rH_{threshold(k1)}, and rH_{threshold(k2)}, respectively, are rH values provided in the form of “% rH.”5. The method according to claim 4, wherein rH_{threshold(k)}, is an rH value selected from one of 15, 30, 45, 60 and 75% rH; or wherein rH_{threshold(k1)} is an rH value selected from one of 15, 20, 25, 30, 35, and 40% rH, and rH_{threshold(k2)} is an rH value selected from one of 50, 55, 60, 65, 70, and 75% rH.

6. The method according to claim 1, wherein the determining the concentration of the analyte in step iii) comprises determining at least one relative humidity correction rH_{corr} and/or at least one relative humidity correction function rH_{corr-fct}, independently from one another, in each case based on one of the applicable ranges of relative humidity rH_{appl-range(n)} selected in step ii), and taking into account said at least one relative humidity correction rH_{corr} and/or said at least one relative humidity correction function rH_{corr-fct} for the determining of the concentration of the analyte.

7. The method according to claim 6, wherein one or more of the applicable ranges of relative humidity rH_{appl-range(n)} are associated with their own relative humidity correction rH_{corr} and/or relative humidity correction function rH_{corr-fct}; wherein said relative humidity corrections rH_{corr} and/or relative humidity correction functions rH_{corr-fct} may be selected independently from one another for each applicable range of relative humidity rH_{appl-range(n)}; optionally, wherein at least one of the applicable ranges of relative humidity rH_{appl-range(n)} is not associated with a relative humidity correction rH_{corr} and/or relative humidity correction function rH_{corr-fct}, such that, for said at least one of the applicable ranges of relative humidity rH_{appl-range(n)}, no correction for relative humidity is taken into account for the determining of the concentration of the analyte.

8. The method according to claim 6, wherein, in step iii), the taking into account said at least one relative humidity correction rH_{corr} and/or said at least one relative humidity correction function rH_{corr-fct} further comprises associating one or more of said relative humidity corrections rH_{corr} and/or of said relative humidity correction functions rH_{corr-fct}, independently from one another, with a pre-defined concentration range of the analyte concentration, said pre-defined concentration range being selected from at least two pre-defined analyte concentration ranges.

9. The method according to claim 8, wherein at least one of the pre-defined concentration ranges of the analyte concentration is not associated to a relative humidity correction rH_{corr} and/or relative humidity correction function rH_{corr-fct}, such that, for said at least one of the pre-defined concentration ranges of the analyte concentration, no correction for relative humidity is taken into account for the determining of the concentration of the analyte in step iii).

10. The method according to claim 1, wherein the method further comprises receiving at least one additional estimate value rH_{add-estimate} of the local relative humidity from at least one of:
a) a remote weather service, via a wireless connection to the mobile device;
b) an external electronic device, comprising an ambient humidity sensor, via a wireless connection to the mobile device; and
c) an ambient humidity sensor located in the mobile device;
and wherein step ii) comprises verifying or adjusting the estimate value rHₑₛₜᵢₘₐₜₑ of the local relative humidity, by taking into account at least one of the additional estimate values rH_{add-estimate},
wherein each of rHₑₛₜᵢₘₐₜₑ and rH_{add-estimate}, respectively, are rH values provided in the form of “% rH.”

11. A mobile device having at least one camera and at least one processor, the mobile device being configured for determining a concentration of an analyte in a bodily fluid, wherein the at least one concentration of the analyte is determined from a color formation reaction at a reagent test region; and wherein the mobile device further is configured for performing at least steps i) to iii) of the computer-implemented analytical method according to claim 1.

12. A kit, comprising the mobile device according to claim 11, and at least one of an optical test element and a color reference card, said optical test element and/or said color reference card being associated with the reagent test region.

13. A computer program comprising instructions which, when the program is executed by the mobile device according to claim 11, cause the mobile device to carry out at least steps i) to iii) of the computer-implemented analytical method according to claim 1.

14. A computer-readable storage medium comprising instructions which, when executed by the mobile device according to claim 11, cause the mobile device to carry out at least steps i) to iii) of the computer-implemented analytical method according to claim 1.
